(19) United States
(12) Patent Application Publication
(10) Pub. No.: US 2024/0207244 A1
(43) Pub. Date: Jun. 27, 2024

(54) COMPOSITIONS AND METHODS FOR TREATING NEURODEGENERATIVE DISORDERS THROUGH INHIBITION OF CD44 AND FERM PROTEIN INTERACTION

(71) Applicant: Arizona Board of Regents on Behalf of the University of Arizona, Tucson, AZ (US)

(72) Inventors: Vijay Gokhale, Tucson, AZ (US); May Khanna, Tucson, AZ (US); Judith Tello Vega, Tucson, AZ (US); Haley Williams, Tucson, AZ (US); Marcel Patek, Tucson, AZ (US); Jake Carlson, Tucson, AZ (US)

(21) Appl. No.: 18/287,427

(22) PCT Filed: Apr. 19, 2022
(86) PCT No.: PCT/US22/25349

§ 371 Date: Oct. 18, 2023

Related U.S. Application Data
(60) Provisional application No. 63/176,702, filed on Apr. 19, 2021.

(51) Int. Cl. A61K 31/4545; A61K 45/06

(52) U.S. Cl. CPC A61K 31/4545; A61K 45/06

(57) ABSTRACT

This invention relates generally to neurodegenerative diseases and conditions (e.g., Alzheimer's disease) characterized with aberrant CD44 and FERM protein interaction. This invention further relates to methods and compositions for treating such neurodegenerative diseases and conditions with pharmaceutical compositions comprising agents capable of inhibiting CD44 and FERM protein interaction.

### CROSS-REFERENCE TO RELATED APPLICATION

This application claims priority to and the benefit of U.S. Provisional Patent Application No. 63/176,702 filed Apr. 19, 2021, which is hereby incorporated by reference in its entirety.

### FIELD OF THE INVENTION

This invention relates generally to neurodegenerative diseases and conditions (e.g., Alzheimer's disease) characterized with aberrant CD44 and FERM protein interaction. This invention further relates to methods and compositions for treating such neurodegenerative diseases and conditions with pharmaceutical compositions comprising agents capable of inhibiting CD44 and FERM protein interaction.

### BACKGROUND OF THE INVENTION

The most common age-related neurodegenerative disorder, Alzheimer's disease (AD), constitute a major public health problem due to an increasingly aging population. Reported deaths from AD increased 146% during the last 20 years. Moreover, the number of people living with AD in the US is projected to increase from more than 5 million to nearly 14 million in 2050. The growing elderly population affected by AD will bring a major financial burden with significant implications for the nation's health and socioeconomic institutions. Nowadays, practically all drug treatments tested for AD have failed to demonstrate any efficacy.

Therefore, novel effective therapeutics approaches to prevent and/or slow the progression of AD are urgently needed.

The present invention addresses this need.

### SUMMARY

This invention relates generally to neurodegenerative diseases and conditions (e.g., Alzheimer's disease) characterized with aberrant CD44 and FERM protein interaction. This invention further relates to methods and compositions for treating such neurodegenerative diseases and conditions with pharmaceutical compositions comprising agents capable of inhibiting CD44 and FERM protein interaction.

In certain embodiments, the present invention provides a composition comprising agents capable of inhibiting CD44 and FERM protein interaction, and/or inhibiting TDP-43. Such compositions are not limited to specific agents capable of inhibiting CD44 and FERM protein interaction. In some embodiments, the agents are capable of inhibiting interaction between CD44 and one or more of FERMT2, Ezrin, Radixin and Moesin. In some embodiments, the agents are capable of inhibiting microglial communication to neurons upregulated in AD related to CD44 and FERM protein interaction. In some embodiments, the agents are capable of inhibiting microglial communication to neurons upregulated in AD related to CD44 and Moesin protein interaction. In some embodiments, the agents are capable of inhibiting the interface of CD44 and FERM protein interaction. In some embodiments, the agents are capable of inhibiting the interface of CD44 and Moesin interaction. In some embodiments, the agents are capable of inhibiting communication between CD44 and FERM proteins. In some embodiments, the agents are capable of inhibiting communication between CD44 and Moesin. In some embodiments, the agent is selected from FERM1-10 or P-8. In some embodiments, the agent has a piperdine-methyl (or similar) chemical structure. In some embodiments, the agent has a N-methyl-pyrazolopyridine (or similar) structure. In some embodiments, the agent has the following structure (or similar):

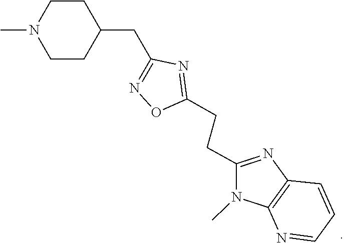 [SMILES: CN1CCC(Cc2noc(CCc3nc4cccnc4n3C)n2)CC1]

In some embodiments, the agent has the following structure (or similar):

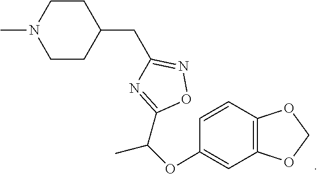 [SMILES: CC(Oc1ccc2c(c1)OCO2)c1nc(CC2CCN(C)CC2)no1]

In some embodiments, the agent is capable of interacting, engaging, and/or binding with one or more of the amino acids shown in FIG. 7. In some embodiments, the agent is a small molecule, an antibody, nucleic acid molecule (e.g., siRNA, antisense oligonucleotide, an aptamer), or a mimetic peptide.

In certain embodiments, the present invention provides a method of treating a mammal suffering from a neurodegenerative disorder comprising administering to the mammal a pharmaceutical composition comprising one or more agents capable of inhibiting CD44 and FERM protein (e.g., Moesin) interaction, and/or inhibiting TDP-43.

In some embodiments, wherein the neurodegenerative disorder is selected from AD, Parkinson's disease, Huntington's disease, amyotrophic lateral sclerosis, motor neuron disease. In some embodiments, the AD is an early stage, prodromal phase of AD.

In some embodiments, the mammal is a human patient.

In certain embodiments, the present invention provides a method for preventing and/or inhibiting neuronal TDP-43 activity in a mammal in need thereof, the method comprising administering to the mammal a composition comprising one or more agents capable of inhibiting TDP-43 activity.

In some embodiments, the composition is capable of protecting neurons from necrosome formation and/or necroptosis activity.

In some embodiments, the mammal is suffering or at risk of suffering from a neurodegenerative disorder selected from AD, Parkinson's disease, Huntington's disease, amyotrophic lateral sclerosis, motor neuron disease.

In some embodiments, the mammal is a human patient.

In certain embodiments, the present invention provides a method for preventing and/or inhibiting neuronal interaction between CD44 and FERM proteins (e.g., Moesin) in a mammal in need thereof, the method comprising administering to the mammal a composition comprising one or more agents capable of inhibiting neuronal interaction between CD44 and FERM proteins (e.g., Moesin).

In some embodiments, the mammal is suffering or at risk of suffering from a neurodegenerative disorder selected from AD, Parkinson's disease, Huntington's disease, amyotrophic lateral sclerosis, motor neuron disease.

In some embodiments, the mammal is a human patient.

### BRIEF DESCRIPTION OF THE DRAWINGS

FIG. 1: CD44 interaction with intracellular Moesin. A. Cartoon modeling of the structure of CD44 binding to Moesin (PDB:) through a short region in the cytoplasm.

FIG. 2A-B: In silico docking of peptide binding site on MSN. A. peptide binding site is shown on electrostatic map of MSN. B. Docking pose of the top binding compound from the peptidomimetic search (compound P1).

FIG. 3: Biophysical characterization of small molecule binding to Moesin (MSN). A. MST (have to put 3 mst here) B. STD-NMR.

FIG. 4A-G: HSQC.

FIG. 5: SV40 cells co-transfected with CD44_S+mCherry Moesin. Maximum intensity projection [40×]. Scale Bar 50 uM.

FIG. 6: A. Locomotor Activity. B. Climbing assay C. Survival curve.

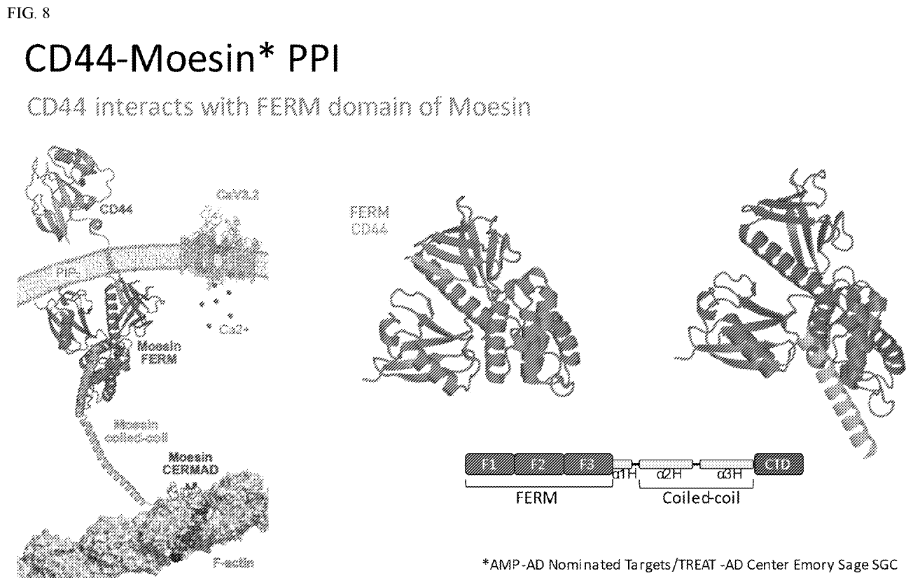

FIG. 7A-B: Testing Derivatives of P1. A. P1 B. P1Sar3.

FIGS. 8-10 show additional data related to the present invention.

### DETAILED DESCRIPTION

Alzheimer's disease (AD) is a progressive and degenerative brain disorder that is the leading cause of dementia. Epidemiological studies have indicated the prevalence rate for AD to be doubling every 5 years and expected to reach 114 million by 2050. Currently, AD is behind cancer and coronary heart disease as the most expensive disorders in the United States. There are currently no cures for Alzheimer's disease.

There is little agreement on what causes this devastating age-related disease. The neurofibrillary tangles (NFTs) and the abnormal extracellular accumulation and deposition of the amyloid-β peptide (Aβ) first described by Alois Alzheimer, are the hallmarks that have been used worldwide as diagnostic criteria for the disease, but whether they are causes or consequences of AD is yet unknown. In addition to the established pathology of NFTs and senile plaques restricted to the neuronal compartment, strong interconnections with immunological mechanisms in the brain are also present, and high expression of inflammatory mediators has been reported. In fact, a network-based integrative approach identified the immune/microglia module as the molecular system most strongly associated with the pathophysiology of AD. Neuroinflammation is now thought to contribute to and aggravate AD pathology. Among the various molecules of the immune system that are associated with AD, two critical targets that are upregulated during AD progression, have emerged, CD44 and FERM proteins. These two targets are critical in neuroinflammation and interact together.

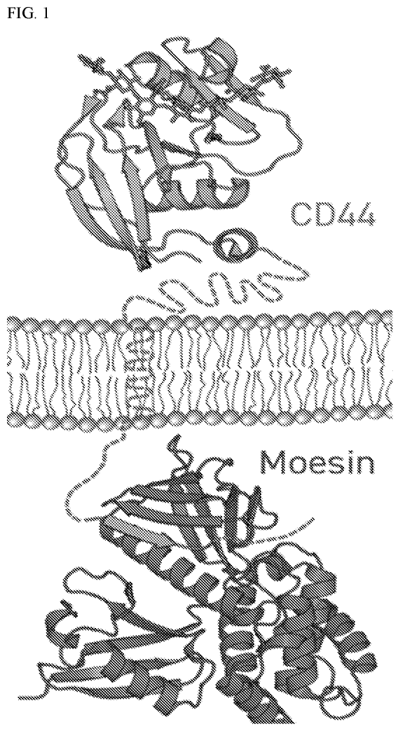

CD44 encodes a multifunctional cell-surface glycoprotein that serves as a receptor for hyaluronic acid of a myeloid cell- or astrocyte/microglial-cell-expressed gene with variants associated with an increased risk of developing AD (FIG. 1). CD44 expression is upregulated on astrocytes from post-mortem samples of human AD brains. Expression of CD44 splice variants and CD44S (which does not contain any alternative exon) were significantly higher in AD patients compared to non-AD controls. Consistently, a large-scale proteomic analysis of AD brain and CSF revealed significant elevations of CD44 associated with microglia and astrocyte activation.

Proteins with a FERM domain (FERMT2, Ezrin, Radixin and Moesin) have roles in structural integrity, transport, and signaling functions. They are found to interact with various proteins at the interface between the plasma membrane and the cytoskeleton. Upregulation of Ezrin was seen in AD subjects, these changes in protein abundance are associated with neurodegeneration not only in humans, but also in a tauopathy mouse model. Another FERM protein, Moesin (Msn), has shown increased expression in the human AD brain and 5×FAD mouse. Interestingly, Msn expression is nearly exclusively found in microglia that surround Aβ plaques in 5×FAD brains.

Both CD44 and Msn were identified as a hub proteins of an inflammatory co-expression module positively associated with AD neuropathological features and cognitive dysfunction. CD44 and FERM proteins interact through an extended cytoplasmic peptide that binds to the FERM proteins intracellularly through a protein/peptide interaction (FIG. 1). Manipulation of CD44 and FERM proteins and/or their respective pathways appears to provide an exciting novel target for the treatment of AD since these proteins are upregulated in AD and predominantly found in microglia. Therefore, our goal is to target the CD44/FERM protein-peptide interaction to mitigate neuroinflammation and stop progression of the disease.

Unlike strategies directly targeting CD44 that cause side effects, the approach implemented in experiments conducted during the course of developing embodiments for the present invention was to target a protein-protein interaction, CD44 binding to Msn, involved in microglia communication to neurons that is upregulated in AD. Because both proteins are upregulated, targeting the interface will disrupt this communication and would downregulate two highly upregulated targets with one interface.

Indeed, such experiments defined the known structural interactions between CD44, and Msn based on a co-crystal structure of this protein-protein complex. It was proposed that the binding of Msn through CD44 could be mimicked by small molecules. By performing in silico docking on the CD44 binding pocket, using a virtual screen of 50,000 compounds and 200,000 pharmacophores and guided by FERM domain-peptide co-crystal structures, such experiments identified druggable pockets in these FERM proteins. Top scoring compounds were tested for binding to recombinant purified FERM proteins using several biophysical methods. Such experiments further indicated that derivatives of the initial compounds retain inhibitory status.

Accordingly, the present invention relates generally to neurodegenerative diseases and conditions (e.g., Alzheimer's disease) characterized with aberrant CD44 and FERM protein interaction. This invention further relates to methods and compositions for treating such neurodegenerative diseases and conditions with pharmaceutical compositions comprising agents capable of inhibiting CD44 and FERM protein interaction.

In certain embodiments, the present invention provides a composition comprising agents capable of inhibiting CD44 and FERM protein interaction, and/or inhibiting TDP-43. Such compositions are not limited to specific agents capable of inhibiting CD44 and FERM protein interaction. In some embodiments, the agents are capable of inhibiting interaction between CD44 and one or more of FERMT2, Ezrin, Radixin and Moesin. In some embodiments, the agents are capable of inhibiting microglial communication to neurons upregulated in AD related to CD44 and FERM protein interaction. In some embodiments, the agents are capable of inhibiting microglial communication to neurons upregulated in AD related to CD44 and Moesin protein interaction. In some embodiments, the agents are capable of inhibiting the interface of CD44 and FERM protein interaction. In some embodiments, the agents are capable of inhibiting the interface of CD44 and Moesin interaction. In some embodiments, the agents are capable of inhibiting communication between CD44 and FERM proteins. In some embodiments, the agents are capable of inhibiting communication between CD44 and Moesin. In some embodiments, the agent is selected from FERM1-10 or P-8. In some embodiments, the agent has a piperdine-methyl (or similar) chemical structure. In some embodiments, the agent has a N-methyl-pyrazolopyridine (or similar) structure. In some embodiments, the agent has the following structure (or similar):

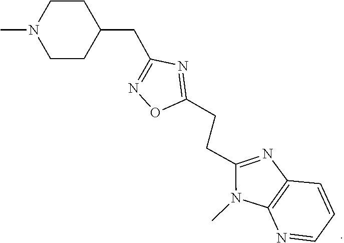 [SMILES: CN1CCC(Cc2noc(CCc3nc4cccnc4n3C)n2)CC1]

In some embodiments, the agent has the following structure (or similar):

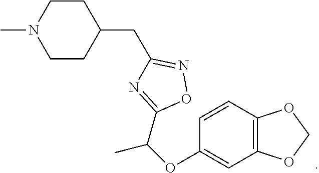 [SMILES: CC(Oc1ccc2c(c1)OCO2)c1nc(CC2CCN(C)CC2)no1]

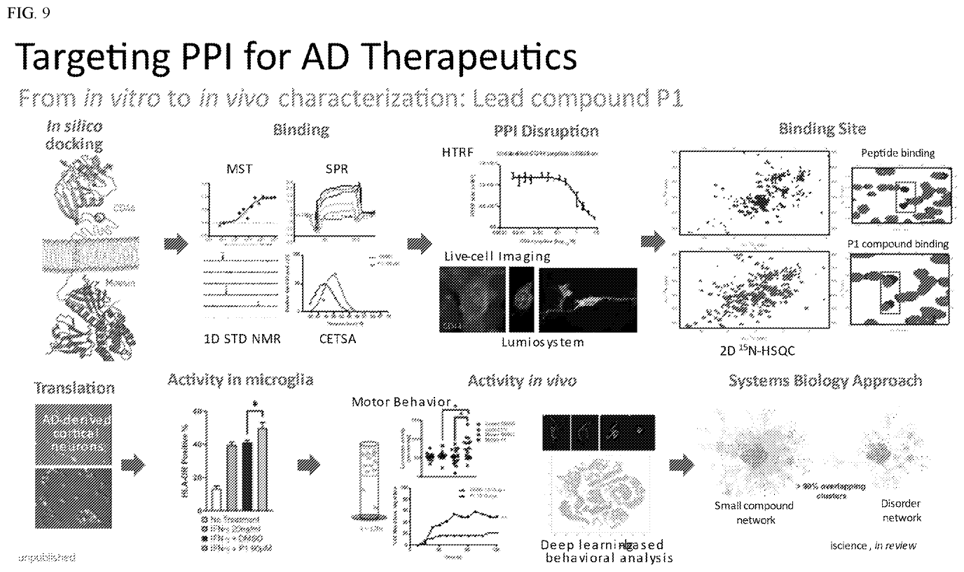

In some embodiments, the agent is capable of interacting, engaging, and/or binding with one or more of the amino acids shown in FIG. 7. In some embodiments, the agent is a small molecule, an antibody, nucleic acid molecule (e.g., siRNA, antisense oligonucleotide, an aptamer), or a mimetic peptide.

In certain embodiments, the present invention provides a method of treating a mammal suffering from a neurodegenerative disorder comprising administering to the mammal a pharmaceutical composition comprising one or more agents capable of inhibiting CD44 and FERM protein (e.g., Moesin) interaction, and/or inhibiting TDP-43.

In some embodiments, wherein the neurodegenerative disorder is selected from AD, Parkinson's disease, Huntington's disease, amyotrophic lateral sclerosis, motor neuron disease. In some embodiments, the AD is an early stage, prodromal phase of AD.

In some embodiments, the mammal is a human patient.

In certain embodiments, the present invention provides a method for preventing and/or inhibiting neuronal TDP-43 activity in a mammal in need thereof, the method comprising administering to the mammal a composition comprising one or more agents capable of inhibiting TDP-43 activity.

In some embodiments, the composition is capable of protecting neurons from necrosome formation and/or necroptosis activity.

In some embodiments, the mammal is suffering or at risk of suffering from a neurodegenerative disorder selected from AD, Parkinson's disease, Huntington's disease, amyotrophic lateral sclerosis, motor neuron disease.

In some embodiments, the mammal is a human patient.

In certain embodiments, the present invention provides a method for preventing and/or inhibiting neuronal interaction between CD44 and FERM proteins (e.g., Moesin) in a mammal in need thereof, the method comprising administering to the mammal a composition comprising one or more agents capable of inhibiting neuronal interaction between CD44 and FERM proteins (e.g., Moesin).

In some embodiments, the mammal is suffering or at risk of suffering from a neurodegenerative disorder selected from AD, Parkinson's disease, Huntington's disease, amyotrophic lateral sclerosis, motor neuron disease.

In some embodiments, the mammal is a human patient.

The methods and compositions of the present invention are useful in treating mammals. Such mammals include humans as well as non-human mammals. Non-human mammals include, for example, companion animals such as dogs and cats, agricultural animals such live stock including cows, horses and the like, and exotic animals, such as zoo animals.

Administration can be by any suitable route of administration including buccal, dental, endocervical, intramuscular, inhalation, intracranial, intralymphatic, intramuscular, intraocular, intraperitoneal, intrapleural, intrathecal, intratracheal, intrauterine, intravascular, intravenous, intravesical, intranasal, ophthalmic, oral, otic, biliary perfusion, cardiac perfusion, priodontal, rectal, spinal subcutaneous, sublingual, topical, intravaginal, transermal, ureteral, or urethral. Dosage forms can be aerosol including metered aerosol, chewable bar, capsule, capsule containing coated pellets, capsule containing delayed release pellets, capsule containing extended release pellets, concentrate, cream, augmented cream, suppository cream, disc, dressing, elixer, emulsion, enema, extended release fiber, extended release film, gas, gel, metered gel, granule, delayed release granule, effervescent granule, chewing gum, implant, inhalant, injectable, injectable lipid complex, injectable liposomes, insert, extended release insert, intrauterine device, jelly, liquid, extended release liquid, lotion, augmented lotion, shampoo lotion, oil, ointment, augmented ointment, paste, pastille, pellet, powder, extended release powder, metered powder, ring, shampoo, soap solution, solution for slush, solution/drops, concentrate solution, gel forming solution/drops, sponge, spray, metered spray, suppository, suspension, suspension/drops, extended release suspension, swab, syrup, tablet, chewable tablet, tablet containing coated particles, delayed release tablet, dispersible tablet, effervescent tablet, extended release tablet, orally disintegrating tablet, tampon, tape or troche/lozenge.

Intraocular administration can include administration by injection including intravitreal injection, by eyedrops and by trans-scleral delivery.

Administration can also be by inclusion in the diet of the mammal such as in a functional food for humans or companion animals.

It is also contemplated that certain formulations containing the compositions capable of inhibiting CD44 and FERM protein (e.g., Moesin) interaction are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methylcellulose, methyl- and propylhydroxy benzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated such as to provide rapid, sustained, or delayed release of the active ingredients after administration to the patient by employing procedures well known in the art. The formulations can also contain substances that diminish proteolytic degradation and promote absorption such as, for example, surface-active agents.

The specific dose can be calculated according to the approximate body weight or body surface area of the patient or the volume of body space to be occupied. The dose will also depend upon the particular route of administration selected. Further refinement of the calculations necessary to determine the appropriate dosage for treatment is routinely made by those of ordinary skill in the art. Such calculations can be made without undue experimentation by one skilled in the art in light of the activity in assay preparations such as has been described elsewhere for certain compounds (see for example, Howitz et al., Nature 425:191-196, 2003 and supplementary information that accompanies the paper). Exact dosages can be determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

The present invention also provides kits comprising agents capable of inhibiting CD44 and FERM protein (e.g., Moesin) interaction and instructions for administering the agent to an animal (e.g., a human patient suffering from a neurodegenerative disorder (e.g., AD)). The kits may optionally contain other therapeutic agents.

### EXPERIMENTAL

The following examples are provided to demonstrate and further illustrate certain preferred embodiments of the present invention and are not to be construed as limiting the scope thereof.

Unlike strategies directly targeting CD44 that cause side effects, the approach implemented in experiments conducted during the course of developing embodiments for the present invention was to target a protein-protein interaction, CD44 binding to Msn, involved in microglia communication to neurons that is upregulated in AD. Because both proteins are upregulated, targeting the interface will disrupt this communication and would downregulate two highly upregulated targets with one interface.

Indeed, such experiments defined the known structural interactions between CD44, and Msn based on a co-crystal structure of this protein-protein complex. It was proposed that the binding of Msn through CD44 could be mimicked by small molecules. By performing in silico docking on the CD44 binding pocket, using a virtual screen of 50,000 compounds and 200,000 pharmacophores and guided by FERM domain-peptide co-crystal structures, such experiments identified druggable pockets in these FERM proteins. Top scoring compounds were tested for binding to recombinant purified FERM proteins using several biophysical methods. Such experiments further indicated that derivatives of the initial compounds retain inhibitory status.

Targeting CD44/FERM in silico. The structure of the Moesin FERM domain complexed with a CD44 cytoplasmic peptide was solved using X-ray crystallography (PDB code: 6TQX)). Molecular docking studies were performed using Schrodinger's Glide docking suite to create a localized grid around the CD44 binding site of human moesin. A 10 Å³ docking grid was generated using the mouse radixin crystal structure centered around the CD44 peptide binding site (residues Arg 248, Ser 251, and Phe252). Virtual screening was performed following a stepwise virtual screening protocol with Glide's Virtual Screening Workflow program. Two different approaches were done for virtual screening: 1. Direct docking to the pocket where the peptide binds and 2. Peptidomimetic search against the CD44 peptide that binds to Moesin (MSN). For the direct docking experiment, a small molecule library of 50,000 compounds from Chembridge Inc., was used as an input for virtual screening and an output 0.1% of the best docking poses were generated. Resulting docking poses were ranked using docking scores and the top 10 compounds (FERM1-10) were selected for further experimental screening. The second pharmacomietic experiment from the same library had similar chemical features as the CD44 peptides based on important interactions as predicted by the crystal structure: the top compounds were selected for further experimental screening (P1-8).

### Biophysical Characterization of Small Molecule Binding

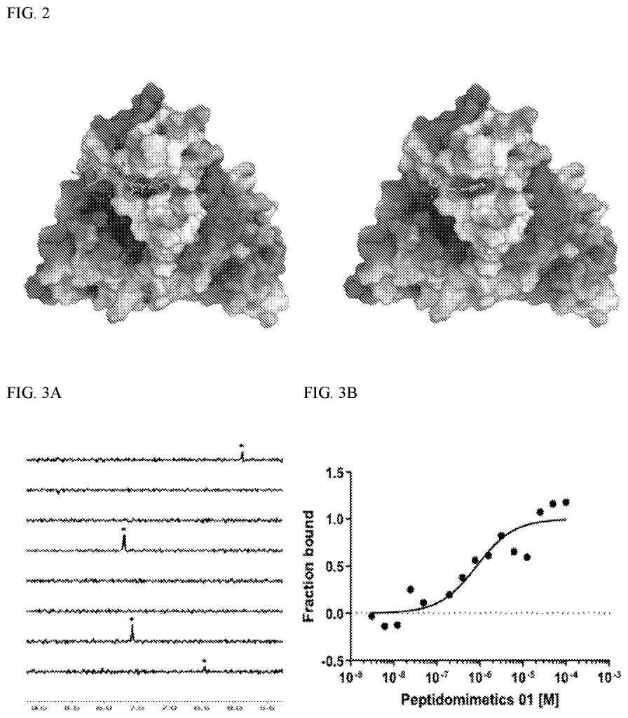

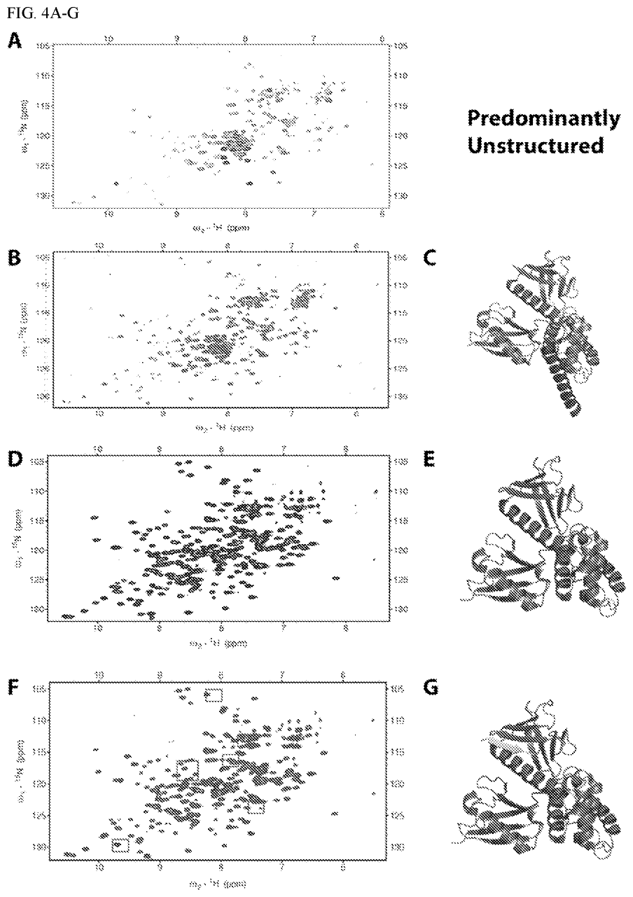

Following in silico docking, we (the inventors) triage binding using saturation transfer difference NMR (STD-NMR) as is routinely done in our laboratory to define binding of small molecules (see, Mollasalehi, N. et al. ACS Chem Biol, doi: 10.1021/acschembio.0c00494 (2020): Francois-Moutal, L. et al. ACS Chem Biol 14, 2006-2013, doi: 10.1021/acschembio.9b00481 (2019): François-Moutal, L. et al. ACS Chem Biol 13, 3000-3010, doi:10.1021/acschembio.8b00745 (2018)) (FIG. 2A). After testing the ability of all compounds to bind to Moesin. One compound, P1, emerged as binding specifically to Moesin, but did not bind to the control GST. We then measured the affinity of binding using micro-scale thermophoresis (MST), a technique to quantify biomolecular interactions, based on thermophoresis. Concentration-dependent measurements were done using MST to determine dissociation constant for binding of P1 to Moesin and we obtained Apparent Kd=Xxx±xxx μM. Data are presented as mean±SD (n=3) (FIG. 3).

Surface Plasmon Resonance (SPR) was used for measuring binding of small molecules as well as disruption of binding of CD44 peptide.

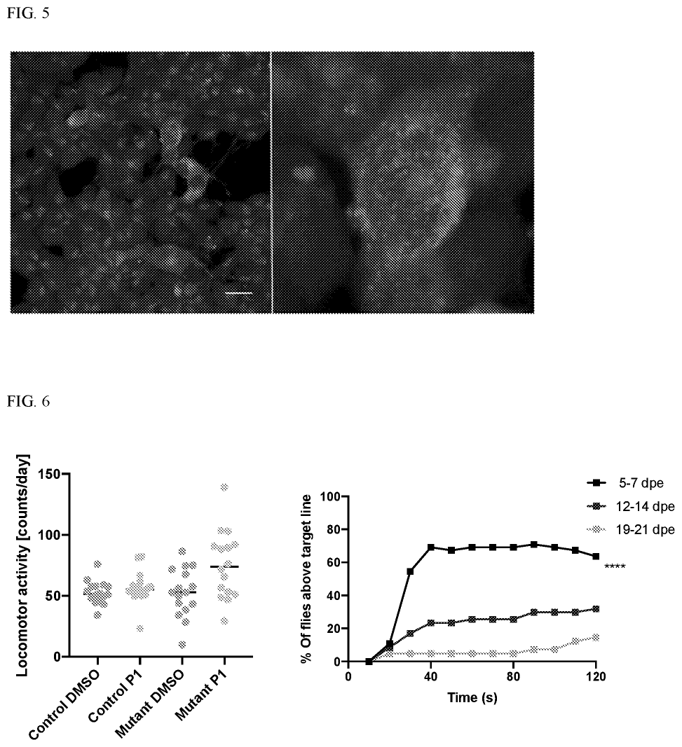

In order to map the binding site of the small molecules, we have purified different constructs of MSN that showed a well folded protein as shown by a 2D ¹⁵N-HSQC. Full length MSN was purified and the 2D ¹⁵N-HSQC did not show a well folded protein (FIG. 4A). We optimized pH, salt and different purification protocols. However, the only condition that yielded a folded protein was following addition of 100 mM Calcium (FIG. 4B, C). Although the protein was well folded, there was still a large region of overlap near 8 ppm: peaks in this region indicating unfolded or structurally similar peaks. A long alpha helix on the C-terminal of MSN (FIG. 4C) did not seem to affect the binding of the CD44 peptide but could potentially cause a clustering of peaks in the HSQC. The protein was cloned in the absence of that helix and the 2D ¹⁵N-HSQC showed significant improvement (FIG. 4D, E). Additionally, the short construct of MSN (shMSN) did not require calcium for folding: the high concentration of calcium could be problematic when screening for small molecule binding. We are currently acquiring 3D NMR experiments to assign all peaks in the HSQC. In the interim, we added CD44 peptide to the 2D ¹⁵N-HSQC of MSN and were able to define which peaks were shifted (or disappeared) due to binding with CD44 (FIG. 4F, G). Based on these shifted peaks, we can now use this as a screening method to define small molecules that bind in the same region as the peptide. We will compare the 2D ¹⁵N-HSQC of the short MSN construct with peptide and with compounds.

### Defining Binding in Cells

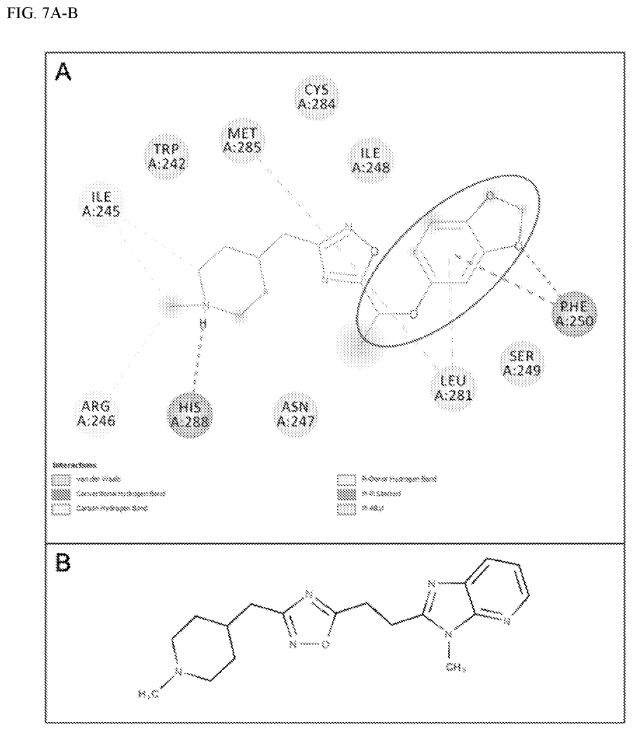

Once we define the binding using purified protein, we validate the binding in cells. We have done this through imaging by using the Lumio™ system for site-specific fluorescent labeling of recombinant proteins in living cells, flow cytometry assays, and cellular thermal shift assay (CETSA).
Lumio™ System Imaging: This recently developed method relies on FLASH (Fluorescein Arsenical Hairpin) technology, which uses biarsenical labeling reagents to bind and detect proteins containing a tetracysteine (TC) motif (Griffin et al., 1998). The biarsenical labeling reagents are nonfluorescent until they bind the TC motif at which time, they become highly fluorescent. Both, a genetically engineered CD44 containing a TC motif, and mCherry-Moesin, were co-transfected into Immortalized Human Microglia (SV40 cell line) (FIG. 5). Ongoing work is being done to optimize this protocol and adapt it for use with small compounds.
Flow Cytometry: Current studies of microglial activation are widely performed to study neuroinflammation and test small compounds and their anti-inflammatory properties. IFNγ was be used to activate macrophage response in HMC3 cells (Human microglial cell line, PMID: 30200996), while flow cytometry was employed in combination with small compounds. HMC3 cells were pre-incubated with either DMSO or P1 for 24 h and subsequently stimulated with IFNγ. Reactive microglia expressing the histocompatibility glycoprotein HLA-DR were detected using flow cytometry. Stimulation of HMC-3 cells with IFNγ caused high expression of HLA-DR and pre-incubation with P1 reduced its level (FIG. 5B).

CETSA: To further ascertain the binding between Moesin and small compounds, we are currently developing Cellular Thermal Shift Assay (CETSA), a convenient cell-based test for identifying target engagement between ligands (i.e. a drug candidate) and their protein targets (PMID:XXX). CETSA has been previously used to measure the binding of small molecules to Ezrin (another FERM protein similar to Moesin) in a cellular environment (PMID: 33003361). The assay consists of the measurement of the shift in thermal stability of the target protein (Moesin) in the presence of the small molecules. The apparent melting temperature (Tm), observed in the presence or the absence of a bioactive compound, can be evaluated by measuring the persistence of soluble protein at different temperatures. If the compound binds to the protein of interest, a shift in melting temperature is observed. A discrete shift in the melting temperature of P1 binding to Moesin was observed in this assay (FIG. 5C).

### Defining In Vivo Efficacy

To assess neuroprotective effects of our compound, we use a Drosophila model of AD. In our phenotype-based small-molecule screening, we measured the age-dependent decline in motor performance (by using both Drosophila activity monitor and climbing assays) and longevity.

### Structure Activity Relationship (SAR) of P1.

Before engaging in custom synthesis, we searched for additional commercially available analogs to expand SAR at the east part of the P1, keeping the piperidinemethyl-heterocycle constant (FIG. 7, red oval). A total of 4.2M compounds were searched for a match to P1 features. “Drug-like” ZINC database 2019 and ChemBridge catalogue provided P1 analogues commercially available. 18 compounds matching the search criteria were selected and tested for biophysical binding. Based on the improved binding involving the N-methylpyrazolopyridine moiety from the first round of analogs (Plsar3), we have proposed several key modifications that will corroborate and expand the binding hypothesis of the P1 series. Our next SAR analogs will thus include structural modifications that will explore hydrogen bond patterns and the size of the fragments in the 5-hydroxypiperonyl/pyrazolopyridine area. Accordingly, structure modifications will consist of five- and six-member heterocycles containing aromatic nitrogens, carbonyl, or hydroxy groups with defined directional vectors. In the next iteration, we intend to explore the importance of the basic group and perhaps charge in the piperidyl area while keeping the central oxadiazole core constant. The ultimate goal of these optimization steps is to identify key pharmacophore features, improve binding affinity, and to keep drug-like properties of our initial hits.

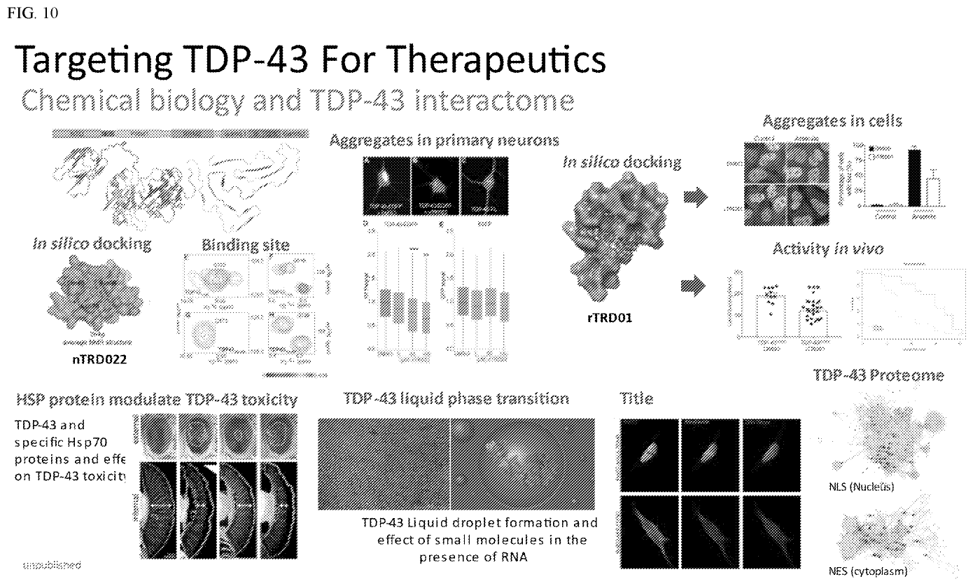

FIGS. 8-10 show additional data related to the invention.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A composition comprising one or more agents capable of inhibiting CD44 and FERM protein interaction, and/or inhibiting TDP-43 activity.

2. The composition of claim 1, wherein the agent is capable of one or more of the following:
inhibiting interaction between CD44 and one or more of FERMT2, Ezrin, Radixin and Moesin;
inhibiting microglial communication to neurons upregulated in AD related to CD44 and FERM protein interaction;
inhibiting microglial communication to neurons upregulated in AD related to CD44 and Moesin protein interaction;
inhibiting the interface of CD44 and FERM protein interaction;
inhibiting the interface of CD44 and Moesin interaction;
inhibiting communication between CD44 and FERM proteins; and
inhibiting communication between CD44 and Moesin.

3. The composition of claim 1, wherein the agent is selected from FERM1-10 or P-8.

4. The composition of claim 1, wherein the agent has a piperdine-methyl (or similar) chemical structure.

5. The composition of claim 1, wherein the agent has a N-methyl-pyrazolopyridine (or similar) structure.

6. The composition of claim 1, wherein the agent has the following structure (or similar):

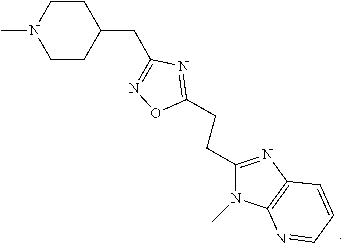 [SMILES: CN1CCC(Cc2noc(CCc3nc4cccnc4n3C)n2)CC1]

7. The composition of claim 1, wherein the agent has the following structure (or similar):

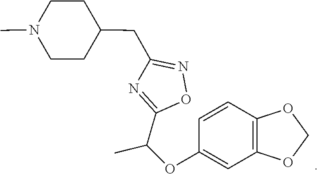 [SMILES: CC(Oc1ccc2c(c1)OCO2)c1nc(CC2CCN(C)CC2)no1]

8. The composition of claim 1, wherein the agent is capable of interacting, engaging, and/or binding with one or more of the amino acids shown in FIG. 7.

9. The composition of claim 1, wherein the agent is a small molecule, an antibody, nucleic acid molecule (e.g., siRNA, antisense oligonucleotide, an aptamer), or a mimetic peptide.

10. A method of treating a mammal suffering from a neurodegenerative disorder comprising administering to the mammal a pharmaceutical composition comprising one or more agents recited in claim 1.

11. The method of claim 1, wherein the neurodegenerative disorder is selected from AD, Parkinson's disease, Huntington's disease, amyotrophic lateral sclerosis, motor neuron disease.

12. The method of claim 11, wherein the AD is an early stage, prodromal phase of AD.

13. The method of claim 1, wherein the mammal is a human patient.

14. A method for preventing and/or inhibiting neuronal TDP-43 activity in a mammal in need thereof, the method comprising administering to the mammal a composition comprising one or more agents recited in claim 1.

15. The method of claim 14, wherein the mammal is suffering or at risk of suffering from a neurodegenerative disorder selected from AD, Parkinson's disease, Huntington's disease, amyotrophic lateral sclerosis, motor neuron disease.

16. The method of claim 14, wherein the mammal is a human patient.

17. A method for preventing and/or inhibiting neuronal CD44 and FERM protein interaction in a mammal in need thereof, the method comprising administering to the mammal a composition comprising one or more agents recited in claim 1.

18. The method of claim 17, wherein the mammal is suffering or at risk of suffering from a neurodegenerative disorder selected from AD, Parkinson's disease, Huntington's disease, amyotrophic lateral sclerosis, motor neuron disease.

19. The method of claim 17, wherein the mammal is a human patient.

20. A composition comprising one or more agents capable of inhibiting CD44 and Moesin protein interaction, and/or inhibiting TDP-43 activity.
